(19) United States
(12) Patent Application Publication
(10) Pub. No.: US 2015/0238364 A1
(43) Pub. Date: Aug. 27, 2015

(54) EARRING ATTACHABLE EARPLUG

(71) Applicants: Matthew Johnson, San Diego, CA (US); Christine Beavis, San Diego, CA (US)

(72) Inventors: Matthew Johnson, San Diego, CA (US); Christine Beavis, San Diego, CA (US)

(21) Appl. No.: 14/187,529

(22) Filed: Feb. 24, 2014

(51) Int. Cl. A61F 11/12

(52) U.S. Cl. CPC A61F 11/12; A61F 2220/0016; A61F 2250/0029; A61F 2220/0033; A61F 2230/0069

(57) ABSTRACT

A hearing protection device is disclosed. The hearing protection device is comprised of an assembly consisting of an earplug attached to a first end of a flexible leash and an earring attachment barrel attached at a second or distal end of the same flexible leash. In use, the attachment barrel may be easily inserted onto a post or hook of any standard earring wherein the earplug may be allowed to dangle from the earring when worn. When the wearer requires sound suppression, he/she simply inserts the earplug into their ear canal without removing the earplug assembly from their earring. The earplug assembly is easily removed from the ear canal by the wearer simply tugging on the flexible leash. The earplug assembly may be removed from the earrings, and may be washed and reused over and over again or used with another pair of earrings.

### BACKGROUND OF THE INVENTION

1. Field of the Invention

This invention relates generally to a hearing protection device, and more specifically to an earplug combined with an earring attachment barrel that retains the earplug close to the ear even when the earplug is not inserted in the ear canal.

2. Background of the Invention

There are many casual environments where one may experience harmful levels of noise such as concerts, festivals, auto racing and air shows. Many examples of prior art patents exist that address this problem wherein the prior art patents propose individual earplugs that may be inserted into the wearer's ear canals. There are many different materials that have been used for these earplugs such as foam, thermoplastic and silicone. There are also many different designs of the earplug that allow more or less sound suppression.

These earplugs are small enough to fit within the ear canal and are thus prone to loss, misplacement, mishandling (such as dropping them on the ground) and other general difficulties associated with small personal items. Prior art patents exists that address this problem in several ways; by the attachment of the earplugs to one another with a cord, by the attachment of the earplugs to the wearer's eyeglasses or safety glasses (as disclosed and taught in U.S. Pat. Nos. 7,213,916; 7,641,334; and 6,302,111), or by the attachment of the earplugs to a flexible strap (U.S. Pat. No. 5,074,375). However, these examples of prior art patents do not address other issues such as: not all people that attend the aforementioned events wear eyeglasses, and the cord that connects some earplugs can get caught in one's hair.

Others have tried to solve these problems with an assembly of a standard earplug that is attached to an earring permanently by means of a typical metal ring that is found at the end of a chain in many earrings. This system has two major flaws; standard earplugs are often a color and/or shape that is not desirable to the wearer, and it requires the wearer to purchase a whole earring that they may not like the style, design or color.

In addition, many standard earplugs are hard to insert into small ear canals and may also be hard to remove. Therefore, a need exists for a sound suppression device that is comfortable, easy to use, hard to lose and takes into consideration changing fashions. The disclosed invention solves this problem in a novel and unique way.

### SUMMARY OF THE INVENTION

A hearing protection device is disclosed. The hearing protection device is comprised of an assembly consisting of an earplug attached to a flexible leash at a first end and an earring attachment barrel attached to the other side or distal end of the flexible leash. In use, the attachment barrel may be easily inserted onto a post or hook of any standard earring wherein the earplug may be allowed to dangle from the earring when worn. When the wearer requires sound suppression, he/she simply inserts the earplug into their ear canal without removing the assembly from their earring. The earplug is easily removed from the ear canal by tugging on the flexible leash. The assembly may be removed from the earrings, and may be washed and reused over and over again or used with another pair of earrings.

It is an object of the present invention to provide an earplug assembly that permits a sound suppression earplug to be quickly and easily removed from an ear canal when the earplug is not needed.

It is still another object of the present invention to provide an earplug which is convenient to store and handle, and is not easily lost.

These and other objects are achieved according to the present invention by combining an earplug with an earring attachment barrel that retains the earplug to the ear even when the earplug is withdrawn from the ear canal itself.

These and other objects and advantages which will become subsequently apparent in the details of construction and operation as more fully hereinafter described in the following specification and the accompanying drawings.

### BRIEF DESCRIPTION OF THE DRAWINGS

Further objects and advantages of the present invention will become apparent as the following description of the illustrative embodiments takes place, in which:

FIG. 1 is a perspective view of the earring attachable earplug assembly in accordance with the present invention; and

FIGS. 2 through 5 are a drawings illustrating different preferred embodiments for attaching the earplug assembly shown in FIG. 1 to an ear lobe.

### DESCRIPTION OF A PREFERRED EMBODIMENT

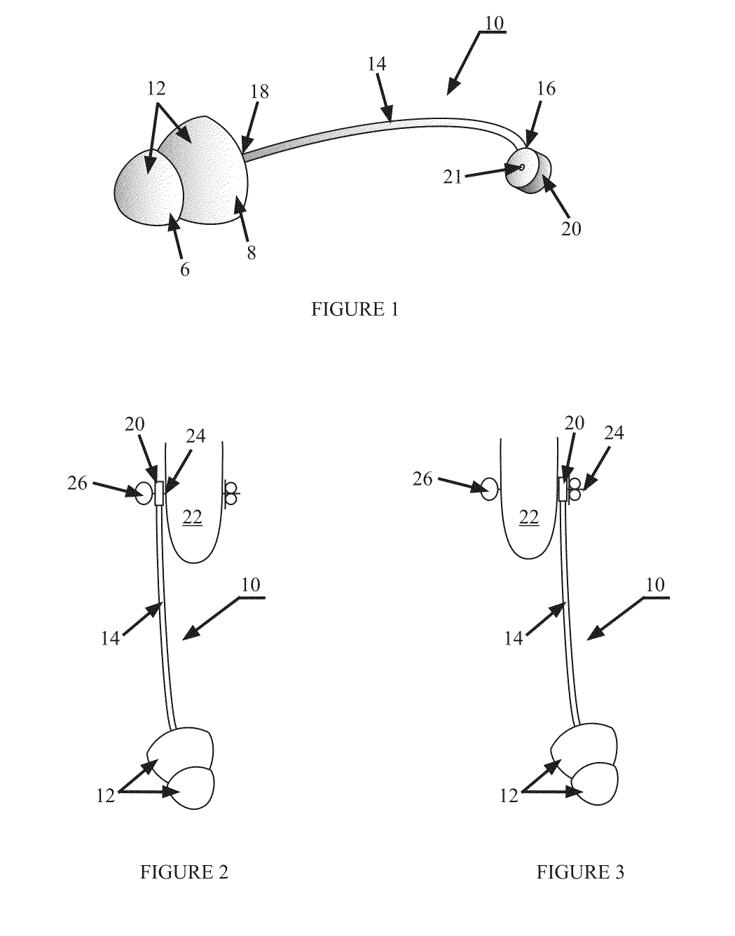

Referring now to FIGS. 1 through 3, there is shown a hearing protection device or earplug assembly 10 in accordance with the present invention. Turning once again to FIGS. 1 through 3, the hearing protection device 10 is an assembly consisting of an earplug 12 attached to a first end 18 of a flexible leash 14 and an earring attachment barrel 20 attached at a second or distal end 16 of the same flexible leash 14. In use, the attachment barrel 20 may be easily inserted onto a post 24 or hook 25 (FIGS. 4 and 5) of any standard earring 26 wherein the earplug 12 may be allowed to dangle from the earring 26 associated with the wearers earlobe 22 when worn. When the wearer (not shown) requires sound suppression, he/she simply inserts the earplug 12 into their ear canal (not shown) without having to remove the earplug assembly 10 from their earring 26. In summary, the hearing protection device 10 is a combination of three interconnected components, namely, an earplug 12, flexible leash 14 and attachment barrel 20, as shown in FIGS. 1. The earplug end 12 of the assembly is chiefly for suppressing sound. This end may be inserted into the user's ear canal at the user's discretion. The earplug 12 is both soft and tapered to allow for easy insertion and comfort during use as is known in the sound suppression arts.

An advantage of using a pliable or flexible leash 14 is to allow the wearer to connect the earplugs 12 to their earrings 26 so that they may hang from the earring 26 until the wearer requires sound suppression. The flexible leash 14 also serves as a convenient and safe method of removing the earplug 12 from the ear canal (not shown). Therefore, it should be understood that the leash 14 that connects the earplug 12 to the earring attachment barrel 20 must be both flexible and strong enough to allow a wearer to pull on it to remove the earplug 12. The leash 14 may be a variety of lengths such that the user is able to have the earplug 12 inserted fully into the ear canal while the earring attachment barrel 20 end of the assembly is still attached to the user's earring 26.

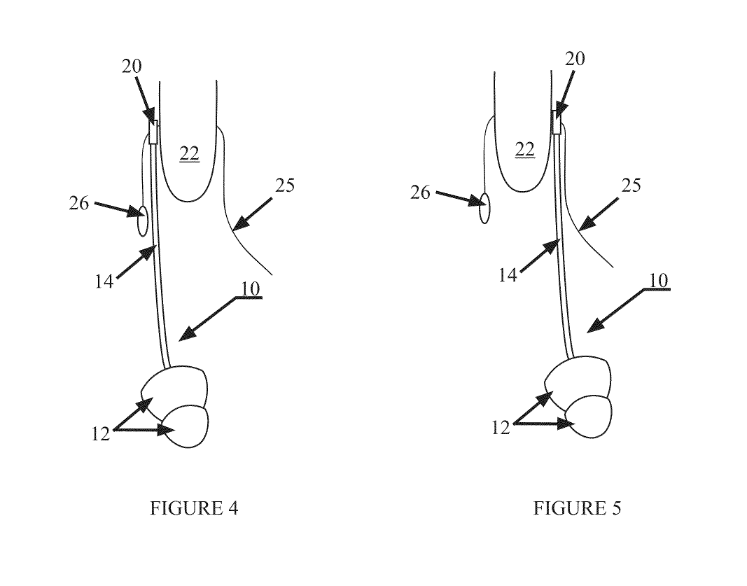

Turning once again to FIGS. 1 through 5, the attachment barrel 20 has an aperture 21 that allows a standard post 24 or hook 25 style earring 26 to be inserted. The aperture 21 is sized such that the assembly may hang from the earring 26 without falling off, but that it is easily removed by pinching the barrel 20 gently and pulling it off the post 24 or hook 25. Referring now to FIGS. 2 through 5, it is shown that the hearing protection device 10 may be applied to the earring in several ways. If the device 10 is worn in front of the ear, then the user will insert the barrel end 20 onto the earring post 24 or hook 25 prior to inserting the earring through the earlobe, as shown in FIGS. 2 and 4. If the device 10 is worn behind the ear, the user will insert the barrel end onto the earring after the earring is in place in the earlobe, as shown in FIGS. 3 and 5. Both options allow for the assembly to stay attached to the earring whether or not the earplug end is inserted into the ear canal.

The device will come as a pair (one for each ear). They may come in a variety of colors and sizes and may be one of a variety of materials including, but not limited to thermoplastic or silicone.

As shown throughout all the FIGURES, the earplug has two parabolic shaped conical flanges, 6 and 8 respectively, attached to a cylinder that reduces in size to become the leash 14. By way of example but not of limitation, single or multiple flanges may compose the earplug, and their shape may vary. Also, the center cylinder may be a variety of sizes and lengths. Lastly, the way in which the cylinder reduces in size to become the leash may also vary.

Although particular embodiments of the invention have been shown and described in full here, there is no intention to thereby limit the invention to the details of such embodiments. On the contrary, the intention is to cover all modifications, alternatives, embodiments, usages and equivalents of the subject invention as fall within the spirit and scope of the invention, specification, and the appended claims.

What is claimed is:

1. The hearing protection device which utilizes a user's earlobe having an earring with a post for sound suppression at the user's ear canal, comprising:
a flexible leash having a first and a second end;
an earplug attached to said first end of said flexible leash; and
an earring attachment barrel attached to said second end of said flexible leash; wherein said earring attachment barrel is attached to an earring located on the user's earlobe, wherein sound suppression is achieved when said earplug is inserted into the users ear.

2. The device according to claim 1, wherein the device is made of one continuous piece including plug, leash and attachment point.

3. The device according to claim 2, wherein said attachment point is a barrel, said barrel defining an aperture that is sized such that a standard earring of either post or hook type may be inserted and removed, but that will allow the device of claim 1 to hang from said earring without falling off.

4. The device according to claim 1, wherein said leash is any length that allows said earplug to be inserted into the ear canal while said leash is still attached to the earring.
